(19) United States
(12) Patent Application Publication
(10) Pub. No.: US 2022/0379089 A1
(43) Pub. Date: Dec. 1, 2022

(54) TARGETED SUCTION CATHETER

(71) Applicant: Orlando V. Morejon, Palmetto Bay, FL (US)

(72) Inventor: Orlando V. Morejon, Palmetto Bay, FL (US)

(21) Appl. No.: 17/828,931

(22) Filed: May 31, 2022

Related U.S. Application Data
(60) Provisional application No. 63/194,666, filed on May 28, 2021.

(51) Int. Cl. A61M 25/01; A61M 25/00

(52) U.S. Cl. CPC A61M 25/0105; A61M 25/0068; A61M 25/008; A61M 2025/091

(57) ABSTRACT

A suction catheter assembly structured to be movably positioned within a lumen of the body including an elongated base having a channel extending interiorly along the length thereof and further structured to connect the channel to a source of negative pressure. A tip is fixedly or removably connected to a distal end of said base and includes an inlet port and a flow path disposed in fluid communication with the channel. A viewing structure is connected to and movable with the base and is oriented relative thereto to define a line of sight providing observation of a path of travel of said base within the body lumen or medical tubing and is disposed laterally outward from at least a majority of a length of said flow path and the inlet port in substantially aligned, parallel relation to a central axis of said channel.

The present application is based on, and a claim of priority is made under 35 U.S.C. Section 119(e) to a provisional patent application that is currently pending in the U.S. Patent and Trademark Office, namely, that having Ser. No. 63/194,666 and a filing date of May 28, 2021, and which is incorporated herein by reference.

### BACKGROUND OF THE INVENTION

### Field of the Invention

This invention is directed to a suction catheter structured to be movably disposed within a lumen of the body, such as the bronchial area, for purposes of removing secretions and other substances. Accurate positioning for aspiration within the body lumen or medical tubing is facilitated by a viewing structure disposed to define a line of sight within the lumen, along the path of travel of the suction catheter.

### Description of the Related Art

Many patients in a hospital, including patients in an Intensive Care Unit (“ICU”), must be fitted with an artificial airway, such as an endotracheal tube, to facilitate their respiration. Typically, the artificial airway tube comprises an elongate, semi-rigid lumen which is inserted into a patient's nose or throat and projects down into airflow communication with the patient's respiratory system. As such, the patient either directly, or with the aid of a respiratory unit, is able to breathe more effectively through the artificial airway tube. Also, a manifold or connector structure is used to connect the artificial airway or endotracheal tube to a source of breathable gas.

However, after prolonged use of ventilation equipment of the type described herein, is necessary to periodically remove respiratory fluids, such as mucus and other secretions from the patient's respiratory system. Recent studies have determined, however, that the accumulation of dried tracheo-bronchial secretions within the bronchial system effectively decreases the breathing capacity of the patient and thereby significantly increases the work of breathing. Moreover, increasing the work of breathing for the patient necessitates that a higher level of support be provided to compensate, and often results in the patient's intubation period and ICU stay being significantly prolonged. Furthermore, it is also seen that thick secretions on the interior walls of the bronchial system are often the basis for continued infection in the lungs, leading to added morbidity and hospital costs for the intubated patient.

Accordingly, there is a long recognized need to remove such secretions, substances, etc. by typically using a suction catheter. Known and/or prior art suction catheters vary in structure, size and operative performance. However, suction catheters are generally considered to be operative to remove such respiratory secretions. As practiced, a negative pressure is applied to the interior of the suction catheter, which serves to draw secretions out of the patient's respiratory system. In order to overcome the various problems and disadvantages associated with removal of such respiratory secretions, known suction catheter construction includes comparatively soft or flexible body portions which can be of different sizes. However larger suction catheters encounter the problem related to the de-oxygenation of the patient. This involves the concurrent suction of secretions and air. Further, catheters of known construction and configuration work “blindly”. As a result, due to the normally un-symmetrical configuration of the bronchial system, there is a tendency for the catheters to be commonly placed into the primary bronchial branch leading to the right lung. In order to overcome disadvantages and problems of this type known suction catheters and like secretion removal instrumentation include various structural designs, which may or may not result in the effective placement and/or positioning within the tracheal system.

It is also known to use instrumentation, referred to as a bronchoscope. Certain advantages of the bronchoscope include structural and operative features which facilitate the “steering” of the operative distal end thereof into a desired location within the bronchial system. However, such steering mechanisms and operative structures are typically located in a proximal and/or “handle” portion of the bronchoscope. Further, the bronchoscope itself may require a larger overall dimension. It is also recognized that bronchoscopes are usually used for thick or heavy secretions which may not be able to be effectively removed by the commonly structured suction catheter. However, due at least in part to the steering capabilities of the bronchoscope, the suction port associated there with may be smaller. Steering mechanisms of the type described occupy significant space in the overall structure or body of the bronchoscope such that the size, placement and orientation of the suction lumen associated there with may have a tendency to clog or be obstructed.

There have been numerous attempts intended to be operative to facilitate the insertion and retraction of various types of intervention devices such as, but not limited to, the aforementioned suction catheter. However, known attempts of this type, while considered to be at least operative, have not provided an operable system capable of overcoming many of the disadvantages and problems associated with assisted respiration and/or the subsequent removal of the bronchial secretions, substances, etc.

In order to overcome these well recognized disadvantages and problems of the type set forth above it is important to provide improved respiratory equipment that will minimize patient discomfort. In addition, improvement in the equipment and/or operative components of a respiratory device should have sufficient operative versatility to treat patients of different age groups. In recognizing such procedural problems, including the accurate placement or positioning of an improved and proposed suction catheter, steering capabilities should be included. Further, such steering capabilities should preferably be integrated in the structure of the catheter assembly itself, rather than being said a supplemental addition as is commonly present in the aforementioned bronchoscope structures. As a result, steering capabilities associated with the conventional bronchoscope will be overcome by adequately dimensioning and configuring the interior lumen of such an improved and proposed suction catheter assembly.

Yet another feature incorporated into an improved and proposed suction catheter assembly would be the provision of a viewing structure including a camera or light viewing source operatively associated with a light source. Such a viewing structure would facilitate the guidance, positioning, orientation, etc. of the inlet port and associated lumen of an improved suction catheter assembly and the application of negative pressure thereto for the effective and efficient removal of bronchial secretions, as required. Further, such a viewing structure should be structurally and operatively compatible with the intake lumen associated with an improved and proposed suction catheter assembly to the extent of providing a clear and continuously present line of sight along the path of travel of the suction catheter assembly in the body lumen or medical tubing of the patient. Finally, any such improved suction catheter assembly, the type described herein, should also be structurally and operatively reliable, while still remaining cost effective to manufacture and assemble. Such additional advantages will facilitate the widespread use and acceptance throughout the medical profession.

### SUMMARY OF THE INVENTION

The present invention is directed to a suction catheter assembly, which may be selectively moved and positioned within a lumen of the human body including, but not limited to, lumens disposed within or associated with the bronchial area. In more specific terms, the insertion of the suction catheter assembly of the present invention would pass into the interior of a medical tubing such as, but not limited to, endotracheal tubing, wherein such medical/endotracheal tubing itself is disposed within the bronchial area or other portion of the patient's body being cleaned, maintained, suctioned, etc. as such, the suction catheter assembly of the present invention includes an elongated base having an interior channel or passage extending along the length thereof. A proximal end of the base is structured for operative attachment to a fluid source, specifically including a source of vacuum or negative pressure.

The opposite or distal end of the elongated base includes a tip connected thereto, wherein the tip includes an interior flow path disposed in fluid communication with the interior channel of the base. Further, the tip includes an inlet port formed at the distal or outer end thereof and disposed in fluid communication with the channel of the base, via the flow path extending along the length of the tip from and including the inlet port to the distal end of the base. Because the interior channel of the base is connected to a source of negative pressure, the inlet port and flow path of the tip, as well as the interior channel of the base, are in concurrent fluid communication with one another and with source of negative pressure. As a result, suction is developed at the inlet port for the collection of secretions and other debris within the body lumen or medical tubing or more specifically the interior of medical tubing disposed within the bronchial passage or other body part.

One feature of the suction catheter assembly of the present invention is the provision of a viewing structure connected to the base and/or the tip. The viewing structure may include a camera or other device capable of capturing visual images as well as a light source. The light source may take a variety of forms including an LED structure, fiber optic bundle, etc. In addition, the light source is disposed and structured to illuminate the interior of the body lumen or medical tubing or medical tubing and more specifically the path of travel of the base and connected tip, as they are movably positioned within and along the length of the body lumen or medical tubing or medical tubing. Therefore, one advantageous feature of the suction catheter assembly of the present invention is the ability to locate and collect secretions and other substances, through illuminated observation within the interior of the lumen.

In addition, the disposition and orientation of the viewing structure specifically including, but not limited to, a lens and/or viewing aperture associated therewith, is such as to prevent or significantly restrict visual interference. More specifically, the viewing structure and the operative components thereof are disposed on the remainder of the catheter assembly in a location which prevents secretions, collected aspirate or other substances, blocking or otherwise interfering with the line of sight along the body lumen or medical tubing or medical tubing, as generated by the viewing structure. Accordingly, the tip including the inlet port and at least a portion of the interior flow path thereof is connected to the base in a laterally offset disposition relative to a central axis of the interior channel of the base. Further, in embodiments where the viewing structure is connected to the base, such as adjacent a distal end thereof, the viewing structure is cooperatively disposed in a non-interfering relation to the tip and/or inlet port, such as being spaced laterally outward from the tip and/or inlet port.

Moreover, as indicated, the viewing structure is disposed to define a line of sight within the body lumen or medical tubing or medical tubing and along the path of travel of the catheter assembly. Due to the non-interfering location of the viewing structure, the generated line of sight will extend outwardly there from in the non-interfering, laterally spaced relation to the inlet port of the tip and at least a majority of a length the interior flow path of the tip. For purposes of clarity, additional descriptive terms of the location and or orientation of the viewing structure and the generated line of sight include the viewing structure and line of sight being in substantially aligned, parallel relation to the central axis of the interior channel of the base. In more specific terms, at least one embodiment of the catheter assembly comprises the viewing structure mounted on or connected to the base, preferably adjacent to a distal end of the base and interior channel, to which the tip is connected. In such a disposition and orientation, the viewing structure will generate or define a line of sight which is spaced laterally outward from the inlet port, at least a portion of the flow path and in substantially aligned, parallel relation to the interior channel of the base and/or the central axis thereof.

Mounting or connection of the viewing structure, including the aforementioned camera and light source are represented in different operative embodiments of the present invention. Accordingly, the viewing structure may be disposed in an at least partially enclosed position within the body of the base. Such a mounting or connection serves to segregate the viewing structure from the interior channel of the base, as well as the surrounding exterior of the base. Yet another embodiment of the present invention comprises the viewing structure mounted on and or connected, either fixedly or removably, to an exterior of the base. Yet an additional operative embodiment of the catheter assembly of the present invention comprises the viewing structure disposed within the interior channel of the base, but being further disposed, dimensioned and configured to eliminate or significantly restrict interference with the intake and flow of aspirate from the inflow port and flow channel of the tip along the length of the interior channel of the base.

Further, one or more additional embodiments of the catheter assembly of the present invention includes the inlet port being at least partially defined by its surrounding, outer periphery being disposed in a plane which is angled or beveled relative to the central axis of the flow path. The inlet port, including the outer periphery thereof, having such an angled or beveled orientation and/or configuration increasingly facilitates the collection of the secretions, substances, etc. from the interior wall surface of the body lumen or medical tubing or medical tubing. This is accomplished by allowing direct engagement of the beveled or angled periphery of the inlet port of the tip with the interior wall surface of the body lumen or medical tubing or medical tubing. As a result, the periphery of the inlet port may perform a scooping, scraping or other physical type action directly on the secretion, which may be at least loosely and removably attached to the inner wall surface of the body lumen or medical tubing or medical tubing.

However, in cooperation with this angled or beveled orientation of the inlet port, at least a portion of the tip is formed of a flexible, deformable material structured to eliminate or significantly restrict the possibility of any injury to the interior surface of the body lumen or medical tubing. Therefore, the material from which at least the outer or distal end of the tip, contiguous to the inlet port is formed has sufficient flexibility or “softness” to be easily deformed when engaging the interior wall surface of the body lumen or medical tubing and providing the aforementioned physical action on a located secretion, etc. Such flexibility will thereby prevent or significantly restrict the possibility of damage to the interior surfaces of the body lumen or medical tubing, when such scooping or scraping action is performed. It is to be further noted that the above noted flexible material may extend along a majority and/or an entirety of the length of the tip, from the outer periphery of the inlet port inwardly towards the proximal end thereof connected to the base.

Also, at least one embodiment of the catheter assembly of the present invention includes the flexible, deformable material, as described above, extending along at least a portion of the length of the tip, and becoming progressively less flexible or deformable towards the distal end of the base, to which the tip is connected. In addition, in each of the structural modifications defining one or more embodiments of the present invention, the flexible, deformable material includes an inherent “memory” which facilitates the tip including the periphery of the inlet port returning to at an original shape, dimension and intended operative orientation. Moreover, at least some portion of the length of the tip, from and including the outwardly or distally disposed inlet port, towards the distal end of the base, may be formed of a material including the “memory” and flexible, temporarily deformable characteristics of the type set forth above.

Also, it is to be further noted that the orientation of the inlet port, as at least partially defined by the surrounding periphery thereof, may be other than the angled or beveled orientation and still be at least partially formed of the aforementioned flexible, temporarily deformable material. By way of example, additional operative embodiments of the suction catheter assembly of the present invention may include the outer periphery defining the inlet port having a substantially flat orientation. As such the surrounding outer periphery of the inlet port is disposed in a plane which is substantially perpendicular to the central axis of the flow path of the tip.

As at least generally set forth above, factors associated with a successful collection of secretions, etc. from within a body lumen or medical tubing at least partially depends on the accurate locating of the secretions, etc. to be collected. As is recognized in the prior art, the asymmetric configuration or disposition of the primary branches of the lungs tend to direct a catheter to and through the right branch. Therefore, the ability to “steer” the suction catheter assembly of the present invention will render its operation significantly more efficient. As also noted herein, steering factors associated with similar medical instruments such as, but not limited to a bronchoscope, involve relatively complex and or bulky steering capabilities. Furthermore, the components of these steering capabilities result in the limitation of the hollow channel through which secretions are removed.

Therefore, one operative and structural feature of the suction catheter assembly of the present invention includes the ability to position the inlet port of the tip in a desired location on the interior of the body lumen or medical tubing by incorporating steering capabilities which are inherent in the structural features thereof. This eliminates the necessity of having numerous and sometimes complex separate, auxiliary or supplementary steering components of the type normally associated with prior art catheters and/or bronchoscope structures. More specifically, in at least one embodiment, the base of the catheter assembly of the present invention is formed of a material of at least partially rigid and/or semi-rigid construction. More specifically, this material has sufficient rigidity to facilitate steering of the catheter assembly by facilitating a rotation of the length of the base, such as by a rotational positioning force being applied to the proximal end of the base, as the catheter assembly passes through a body lumen. Further, at least the tubular base of the catheter assembly includes sufficient rigidity to facilitate a longitudinal “pushing” of the catheter in order to position it along the length of the body or artificial lumen through which it passes.

Such rotation of the base will result in a rotation of the length of the base about its length and/or longitudinal axis. In cooperation therewith, the laterally outward spacing of at least a portion of the length of the tip and the attendant laterally outward spacing of the inlet port of the tip, will result in an effective rotational pattern of the tip being laterally outward from and relative to the longitudinal axis of the base. Further, the rotational pattern of the tip may extend through 360° thereby facilitating the tip being variably positioned relative to the body lumen or medical tubing and the interior thereof, upon an axial rotation of the base. Therefore, such axial rotation facilitates a steering or preferred rotational and longitudinal displacement of the tip and the inlet port so as to enter a desired branch of the bronchial area. Once positioned therein, axial rotation of the base can be further conducted to affect location of the inlet port of the tip to further facilitate the collection of secretions, substances, etc., aided by an illuminated viewing thereof by the viewing structure.

Yet additional features of the suction catheter assembly of the present invention includes structure associated with maintaining sterility thereof. More specifically, one or more embodiments of the present invention may include an elongated flexible, collapsible material sleeve connected to the catheter assembly. The sleeve and elongated configuration is sufficient to enclose at least a majority of the length of the catheter assembly and a portion thereof which extends into the body lumen or medical tubing. Moreover, prior to use or application of the catheter assembly, the sleeve is connected at one of its ends to a portion of the catheter assembly adjacent to a proximal end of the base. In its initial orientation, prior to use, the length of the sleeve is sufficient to cover and/or enclose an entirety or at least a majority of the length of the base. In at least one embodiment the sleeve extends over and in the enclosing relation to the length of the base as well as the tip. As a result, the sterility of the catheter assembly and in particular the portion thereof that is inserted through an introductory coupler and into the lumen of the body will be maintained in sterile condition, prior to use.

In order to maintain its intended operative features, the opposite or distal end of the sleeve is secured to the aforementioned introductory coupler, wherein the coupler may or may not be attached to and accompany the catheter assembly upon removal from packaging and prior to use. In the alternative, the distal end of the sleeve may be secured to a distal portion of the base and/or may be secured adjacent to an outer or distal portion of the tip, such as in situations where the introductory coupler is not connected to or considered an original part of the catheter assembly. Further, the introductory coupler may assume a variety of different configurations and, as indicated, may be initially or originally connected to the catheter assembly or may be provided independently thereof. In either application, the introductory coupler may still be operatively disposed in interconnecting relation between the catheter assembly and the body lumen or medical tubing, and in an operative position for introducing the tip and base of the catheter assembly into the body lumen or medical tubing.

As such, preferred fixed connections of opposite ends of the sleeve to the catheter and to the introductory coupler, as well as the flexible material from which the sleeve is formed facilitates the inward collapse or compacting, folding of the sleeve upon itself, concurrently to the tip and distal end of the base being introduced through the introductory coupler and into the body lumen or medical tubing. As indicated above, the inherent flexibility of the material from which the elongated sleeve is formed facilitates its “inward” collapse or folding of the sleeve upon itself at the same time the tip and base are passing into and through the introductory coupling. After the catheter assembly has performed the intended drainage procedure from the bronchial area, the withdrawing of the base and the tip from the body lumen or medical tubing, through the introductory coupler, will result in the concurrent expansion and/or elongation of the sleeve into its original elongated configuration. Such elongation of the sleeve will dispose it in the original overlying, enclosing relation to the portion of the base and eventually the tip upon the respective withdrawal thereof from the body lumen or medical tubing and the introductory coupler. As a result, the sleeve will then be disposed in overlying covering and somewhat isolating relation to the base and possibly the tip thereby allowing it to be reused, while maintaining the sterility of the base and tip, except for exposure to the body lumen or medical tubing.

Yet additional structural features of the present invention include the aforementioned introductory coupler including a guide member mounted on the interior thereof. As set forth herein, the tip is disposed in laterally outward or offset relation to the central axis of the base and the flow channel or passage extending along the interior thereof. Due to such an offset orientation of the tip, there may be a tendency for the tip to inadvertently interfere with interior structure and/or an auxiliary or supplementary vent, port or channel formed in the introductory coupler. The disposition, configuration, dimension and overall structure of the guide member will serve to effectively deflect and/or restrict the offset tip from passing through the introductory coupler in the wrong direction. As a result, the offset tip will be restricted from interruptive engagement with the interior structure or supplementary port, vent, etc. of the introductory coupler.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic representation of one embodiment of the suction catheter assembly, of the present invention.

FIG. 1A is a transverse sectional view along line 1A-1A of the embodiment of FIG. 1.

FIG. 1B is a transverse sectional view along line 1B-1B of the embodiment of FIG. 1.

FIG. 1C is a schematic representation of the embodiment of FIGS. 1, 1A and 1B.

FIG. 2 is a longitudinal sectional view in partial cutaway of another embodiment of the suction catheter assembly of the present invention.

FIG. 2A is a transverse sectional view along line 2A-2A of the embodiment of FIG. 2.

FIG. 2B is a transverse sectional view along line 2B-2B of the embodiment of FIG. 2.

FIG. 3 is a longitudinal sectional view of yet another embodiment of the present invention.

FIG. 3A is a longitudinal sectional view of yet another embodiment of the present invention.

FIG. 3B is a longitudinal sectional view of yet another embodiment of the present invention.

FIG. 3C is a perspective view in partial cutaway of yet another embodiment of the present invention.

FIG. 4 is a schematic view of yet another embodiment of the suction catheter assembly of the present invention.

FIG. 5 is a schematic representation of the steering capabilities of the suction catheter assembly of the present invention.

FIGS. 5A and 5B are each schematic representations of a monitor display along a line of sight of the viewing structure of the present invention.

FIG. 6A is an at least partially schematic representation of the catheter assembly of the present invention at least partially maintained in sterile condition prior to use and/or insertion into the lumen of the body of the patient.

FIG. 6B is an at least partially schematic representation of the embodiment of the catheter as represented in FIG. 6A, subsequent to insertion in a body lumen or medical tubing.

FIG. 7A is a schematic representation in partially exploded form of another embodiment of the suction catheter assembly of the present invention.

FIG. 7B is a schematic representation in partially exploded form of the embodiment of FIG. 7A in at least partially assembled form.

FIG. 8 is a schematic representation of one embodiment of the present invention including a trap assembly.

FIG. 9A is a side view in partial cutaway of yet another embodiment of the handle or control hub of the present invention.

FIG. 9B is a top view and partial cutaway, of the embodiment of FIG. 9A.

FIG. 9C is a side interior view in at least partially schematic form of the embodiment of FIGS. 9A and 9B.

Like reference numerals refer to like parts throughout the several views of the drawings.

### DETAILED DESCRIPTION OF THE PREFERRED EMBODIMENT

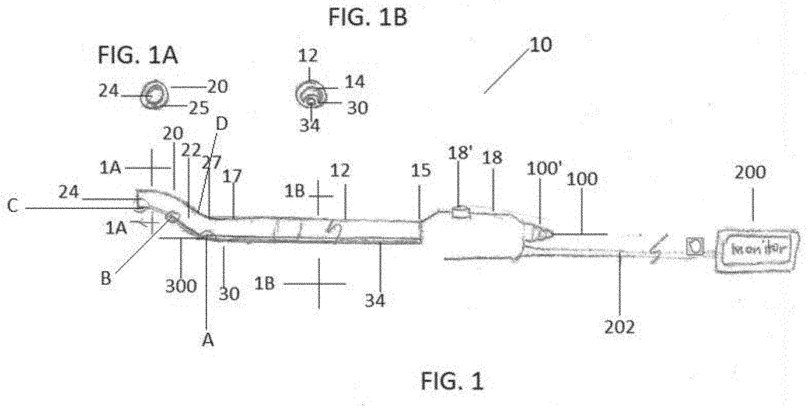

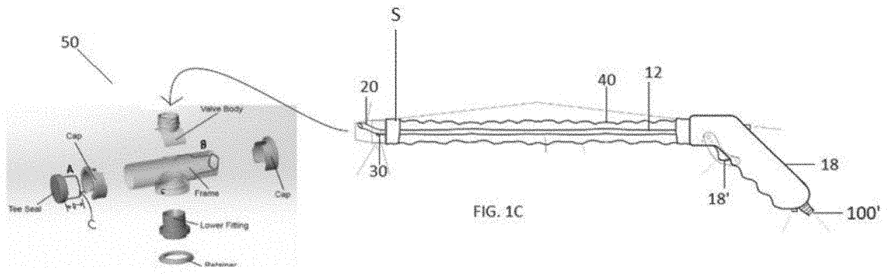

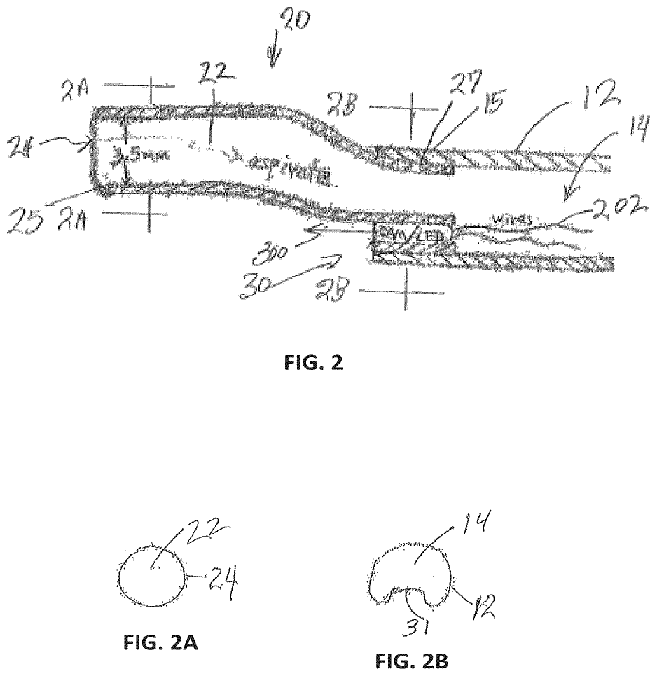

With initial reference to FIGS. 1, 1A, 1B, and 1C, the present invention is directed to a suction catheter assembly, generally indicated as 10 which may be selectively moved and positioned within a lumen of the human body including, but not limited to, lumens associated with the bronchial area. As such, the suction catheter assembly 10 of the present invention includes an elongated base 12 having an interior channel or passage 14 extending along the length thereof (see FIG. 2). The base 12 includes a proximal end 15 and a distal end 17, wherein the proximal end 15 of the base 12 is structured for operative attachment to a handle and/or control hub 18, which in turn is connected in fluid communication with a fluid source, specifically including a source of vacuum or negative pressure as schematically represented as 100, via a fluid port 100′ formed on the handle or control hub 18. Further, the control hub 18 may include a control segment 18′ used to regulate vacuum to and through the channel 14 of the base 12.

As a generally represented in FIG. 1C the catheter assembly 10 is represented in a more conventional form and as utilized, may the introduced into the appropriate lumen via adjustable connector, coupler and or valve assembly generally indicated as 50. In more specific terms, the valve assembly 50 may be of the type disclosed in one or more embodiments of currently pending provisional patent application having Ser. 63/194,664, filed on May 28, 2021.

The opposite or distal end 17 of the base 12 includes a tip generally indicated as 20 connected thereto. As represented in the embodiment of FIG. 1, the tip 20 may be integrally or fixedly connected to the distal end 17 of the base 12. However, as described in greater detail with primary reference to at least FIG. 2, the tip 20 may or may not be removably connected to the distal end 17 of the base 12. In either embodiment, the tip 20 includes an interior flow path 22 (see FIG. 2) disposed in fluid communication with the interior channel 14 of the base 12. Further, the tip 20 includes an inlet port 24 formed at the distal or outer end 25 thereof and disposed in fluid communication with the channel 14 of the base 12, via the flow path 22 extending along the length of the tip 20 from and including the inlet port 24 to the proximal end 15 of the base 12. Because the interior channel 14 of the base 12 is connected to a source of negative pressure 100, the inlet port 24 and flow path 22 of the tip 20, as well as the interior channel 14 of the base 12, are in concurrent fluid communication with one another and with source of negative pressure 100. As a result, suction is developed at the inlet port 24 for the collection of secretions, substances and other debris within the body lumen or medical tubing.

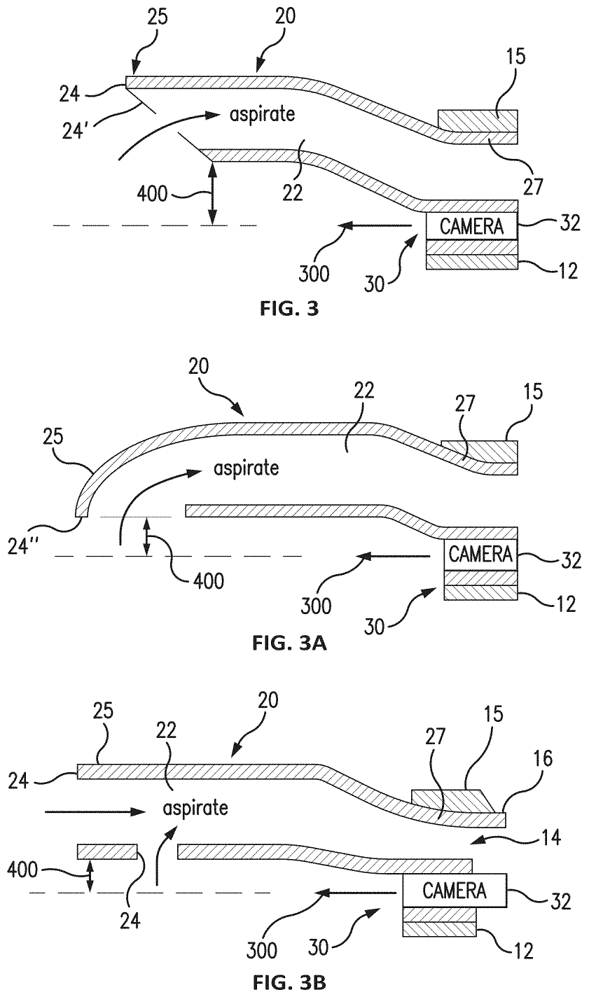

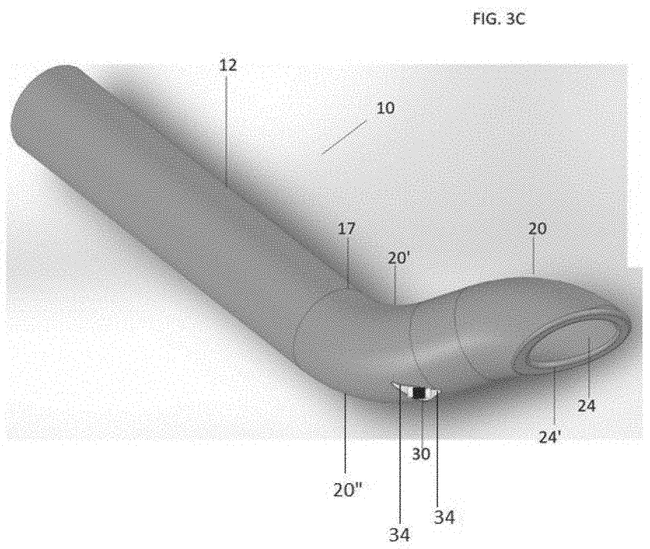

One feature of the suction catheter assembly 10 of the present invention is the provision of a viewing structure, generally indicated as 30, which in the embodiment of FIGS. 1, 2-2B and 3-3C is connected to the base 12 and in alternative embodiments, along the length of the tip 20 itself. The viewing structure 30 may include a camera or other device 32 capable of capturing visual images as well as a light source 34. The light source 34 may take a variety of forms including an LED structure, fiber optic bundle, etc. In addition, the light source 34 is disposed and structured to illuminate the interior of the body lumen or medical tubing and more specifically the path of travel of the base 12 and connected tip 20, as they are movably positioned within and along the length of the body lumen or medical tubing. Therefore, one advantageous feature of the suction catheter assembly 10 of the present invention is the ability to locate and collect secretions and other substances, through illuminated observation within the interior of the lumen. Further, as represented in the embodiment of FIG. 3C, the viewing structure 30 including the light source 34 is positioned along the curved or deflected tip 20 and, more specifically, along the inside radius of the tip 20.

As represented in FIGS. 2A and 2B, the inlet port 24 is appropriately dimensioned to intake secretions, substances, etc. as set forth in greater detail herein. As such, an appropriate dimension for the internal diameter thereof is preferably, but not exclusively, about 3.5 mm to 4.7 mm. Also, the interior channel 14 of the base 12 may be dimensioned to receive the proximal end 27 of the tip 20, in fixed or removable connection thereto. When fixedly attached, the tip 20 may be over molded or insert molded onto the distal end 17 of the catheter base 12. Further the configuration thereof may include a receiving area 31 appropriately disposed and dimensioned for the mounting of the viewing structure 30.

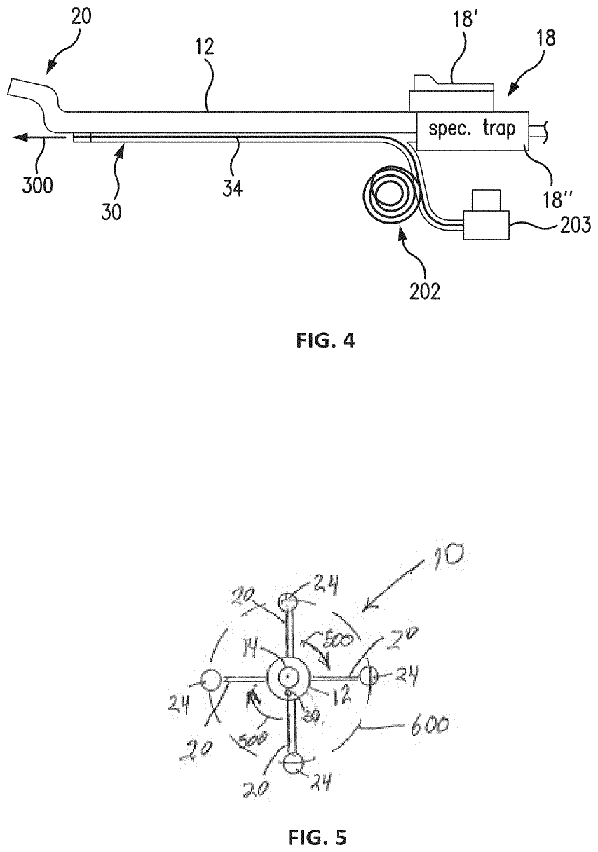

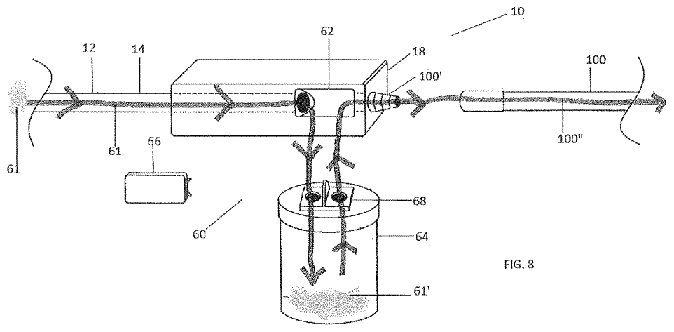

As also represented in FIG. 1, the viewing structure 30 may be operatively connected to a display or monitor 200 via appropriate connections 202. The appropriate operative connections 202 may include hardwiring and/or appropriate connector such as a USB connector or other appropriate connector 203 serving as an interface with the display/monitor 200, as represented in FIG. 4. As also represented therein the control hub or handle 18 may include a trap or collection area for the collected secretions, etc., one embodiment of which is represented in detail hereinafter with reference to FIG. 8.

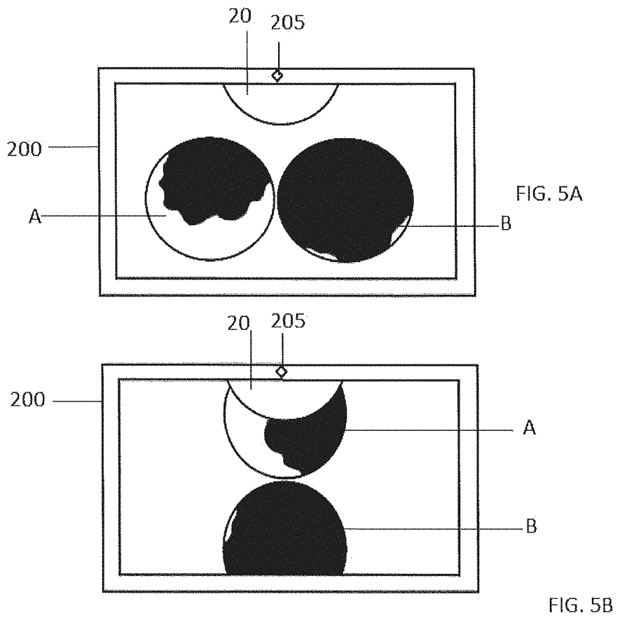

The disposition and orientation of the viewing structure 30, specifically including, but not limited to, a lens and/or viewing aperture (not shown) associated therewith, is such as to prevent or significantly restrict visual interference. More specifically, the viewing structure 30 and the operative components thereof (lens, viewing aperture, etc.) are disposed on the remainder of the base 12 in a location which prevents secretions, collected aspirate or other substances, blocking or otherwise interfering with the line of sight 300 along the body lumen or medical tubing, as generated by the viewing structure 30. Accordingly, the tip 20 including the inlet port 24 and at least a portion of the interior flow path 22 thereof may be connected to the base 12 in a deflected, laterally offset disposition relative to the base 12 and/or a central axis of the interior channel 14. However, the position of said tip 20, relative to said handle or control hub 18 is fixed, concurrent to the deflected, laterally offset relation to the base, thereby enabling an operator to be continuously aware of the position and orientation of the tip 20 relative to said handle or control of 18. As such, a positional marking or other positional indicator may be placed on an exterior portion of the handle or control hub 18 in order to provide an indication of the position, location etc. of the tip when in a body or artificial lumen. Similarly, and/or alternatively, a positional indicator 205 may be placed on a monitor 200 as represented in and described with reference to in FIGS. 5A and 5B.

Further, in embodiments where the viewing structure 30 is connected to the base 12, such as adjacent a distal end 17 thereof, the viewing structure 30 is cooperatively disposed in a non-interfering relation to the tip 20 and/or inlet port 24, such as being spaced laterally outward from the tip 20 and/or inlet port 24 at least a predetermined distance schematically represented as 400 in FIGS. 3-3B. It is noted that the predetermined distance 400 may in fact vary, but in each instance, must be sufficient to eliminate or significantly restrict secretions which are collected through the inlet port 24 and blocking, covering, or otherwise visually interfering with the viewing structure 30.

Moreover, as indicated, the viewing structure is disposed to define a line of sight 300 within the body lumen or medical tubing and along the path of travel of the catheter assembly 10. Due to the non-interfering location of the viewing structure 30 a predetermined, laterally spaced distance 400 from the inlet port 24 and at least a majority of the length of the tip 20, the generated line of sight 300 will extend outwardly from the viewing structure 30 in the non-interfering, laterally spaced relation 400 to the inlet port 24 of the tip 20 and at least a majority of a length the interior flow path 22. For purposes of clarity, additional descriptive terms of the location and or orientation of the viewing structure 30 and the generated line of sight 300 include the viewing structure 30 and line of sight 300 being in substantially aligned, parallel relation to the central axis of the interior channel 14 of the base 12. In more specific terms, at least one embodiment of the suction catheter assembly 10 comprises the viewing structure 30 mounted on or connected to the base 12, preferably adjacent to a distal end 17 to which the tip 20 is connected. In such a disposition and orientation, the viewing structure 30 will generate or define the line of sight 300 which is spaced laterally outward from the inlet port 24, at least a portion of the flow path 22 and in substantially parallel aligned relation to the interior channel 14 of the base 12 and/or the central axis thereof.

Mounting or connection of the viewing structure 30, including the aforementioned camera and light source are represented in different operative embodiments of the present invention. Accordingly, the viewing structure 30 may be disposed in an at least partially enclosed position within the body of the base 12 as represented in at least FIG. 1B. Such a mounting or connection serves to segregate the viewing structure 30 and/or light source 34 from the interior channel 14 of the base 12, as well as the surrounding exterior surface of the base 12. Yet another embodiment of the present invention comprises the viewing structure 30 mounted on and or connected to an exterior of the base 12, as represented in FIGS. 2 and 3-3B. While not specifically represented, an additional operative embodiment of the catheter assembly of the present invention contemplates the viewing structure 30 disposed within the interior channel 14 of the base 12. However, in such a contemplated embodiment, the viewing structure 30 would be disposed, dimensioned and configured to eliminate or significantly restrict interference with the intake and flow of aspirate from the inflow port 24 and flow path 22 of the tip 20 along the length of the interior channel 14 of the base 12.

Alternatively, and as represented in FIG. 1, the viewing structure 30 may be located in various, different locations “A”, “B”, “C”, and/or “D” on tip 20 instead of tubular base 12, wherein in each of the alternative locations the line of sight will be in alignment with the longitudinal axis of the tip 20, regardless of the tip 20 being offset or deflected.

Further, one or more additional embodiments of the suction catheter assembly 10 of the present invention includes the inlet port 24 being at least partially defined by its surrounding, outer periphery 24′ being disposed in a plane which is angled or beveled relative to the central axis of the flow path 22, as represented in FIG. 3. The inlet port 24, including the outer periphery 24′ thereof, having such an angled or beveled orientation and/or configuration increasingly facilitates the collection of the secretions, substances, etc. from the interior wall surface of the body lumen or medical tubing. This is accomplished by allowing direct engagement of the beveled or angled periphery 24′ of the inlet port 24 of the tip 20 with the interior wall surface of the body lumen or medical tubing. As a result, the periphery 24′ of the inlet port 24 may perform a scooping, scraping or other physical type action directly on the secretion, which may be at least loosely and removably attached to the inner wall surface of the body lumen or medical tubing.

However, in cooperation with this angled or beveled orientation of the inlet port 24, as defined by its outer periphery 24′, at least a portion of the tip 20 is formed of a flexible, deformable material structured to eliminate or significantly restrict any damage to the interior surface of the body lumen or medical tubing. Therefore, the material from which at least the outer or distal end 25 of the tip 20, contiguous to the outer periphery 24′ of the inlet port 24 is formed, has sufficient flexibility or “softness” to be easily deformed when engaging the interior wall surface of the body lumen or medical tubing and providing the aforementioned physical action on a located secretion, etc. Such flexibility will thereby prevent or significantly restrict the possibility of damage to the interior surfaces of the body lumen or medical tubing, when such scooping or scraping action is performed. It is emphasized that the above noted flexible material characteristics may extend along a majority and/or an entirety of the length of the tip 20, from the outer periphery 24′ of the inlet port 24 inwardly towards the proximal end 25 thereof connected to the distal end 17 of base 12.

More specifically, in clinical use, a catheter assembly and/or endoscope is structured to travel along the inside contours of natural body cavities or tubes, or through other medical devices (such as endotracheal tubes or other similar tube-like devices). The fixed inner dimensions of the anatomical cavity or the inner dimensions of another medical device through which the endoscope is coursing creates a limitation on the maximal outer diameter of the endoscope that can be used, which in turn places a limitation on the size of the hollow working channel that can be incorporated into an endoscope design. However, many clinical scenarios, such as during a bronchoscopy for the removal of respiratory secretions, would benefit from use of the largest hollow channel possible to facilitate removal of thick, tenacious secretions. These secretions cannot be removed through endoscopes with smaller hollow channels. As such, it would be beneficial to maximize the hollow channel for secretion removal or for the introduction of larger interventional instruments through the hollow channel, while maintaining the outer diameter of the endoscope of sufficiently small size to fit inside the body cavity or other medical device through which the endoscope is introduced. One or more embodiments of the present invention achieves this desirable improvement over other endoscopes.

As recognized, conventional or existing endoscopes are comprised of a tube with a camera at the distal tip, and the tip may be moved relative to the body of the endoscope by way of cables that are attached to the distal tip of the endoscope and attached to a deflection control mechanism at the handle. These cables require their own channels in the endoscope tube, and these channels limit the amount of area within the tube available to maximize the size of the hollow working channel used for suctioning or passage of interventional instruments. The present invention overcomes such problems by eliminating any cables or cords, that run along additional channels in the endoscope tube. As a result, the cross-sectional area for the passage of fluids or instruments is maximized while maintaining an appropriate outer diameter.

Accordingly, rather than having a movable tip as it is conventionally utilized on existing prior art endoscopes the steering of the catheter assembly is facilitated or achieved by virtue of the deflection of the distal tip 20 as described above, connected to the distal end 17 of the base 12 at an angle offset from the central axis of the tubular base 12, as represented in at least FIG. 3C. Due to the flexible material and at least initially deformable construct of the tip 20, such as from polyurethane or other moldable but flexible and at least temporarily deformable material, the tip 20 due to included “memory” characteristics may be at least substantially straightened or otherwise oriented, when sufficient external force is applied, such as when being advanced through another rigid medical tube or when being rotated inside a natural body cavity or tube. Therefore, the aforementioned “memory” characteristics may be accomplished by the formation of the material from which the tip 20 is formed. In the alternative, at least one embodiment of the tip 20 may include the integration of one or more wires, cords, segments or the like on the interior of the tip 20, or otherwise mounted thereon, (not shown for purposes of clarity), which are inherently structured to return the tip 20 to its original offset, deflected, angled orientation subsequent to the removal of the aforementioned exterior force, serving to straighten or otherwise orient the tip 20. Therefore, when under no external stress the aforementioned memory capabilities of the tip 20 facilitate its default to its preferentially deflected or offset angle and may be preferentially aligned with an adjacent or branching natural body passage or space. In this orientation, the operator can advance the endoscope and the deflected or offset tip introduces and then guides the remainder of the endoscope through the passageway of least resistance, which would be the natural anatomy through which the endoscope is intended to proceed. Accordingly, the steering of the catheter assembly 10 in a preferential direction is achieved, without the need or use of independent, auxiliary or supplementary steering structure, by rotating the tube base 12 to varying degrees (see FIG. 5) until the tip 20 and intake port 24 aligns with the path desired by the endoscope operator. This will eliminate the conventional usage of deflecting the endoscope tip through a dedicated control mechanism on the handle, as is the case with current endoscopes. The elimination of the deflection control pull cables and pull cable channels used in conventional endoscopes results in a larger channel or passage 14 for more effective and safer removal of secretions, as intended.

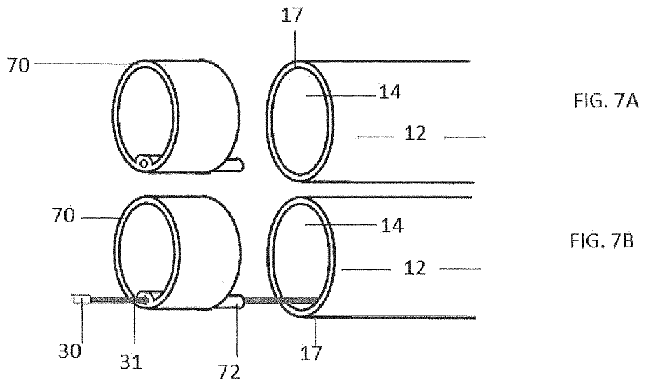

Further, as represented in FIGS. 7A and 7B a single lumen channel 14 is represented within base 12. Further, an end segment 72 may be molded, insert molded and/or over molded onto the distal end 17, and thereby least partially define the distal end 17 of the tubular base 12. The end segment 72 serves to operatively interconnected tip 22 to the remainder of the tubular base 12. Further, the end segment 70 includes a conduit 72 formed on the interior 14, wherein the conduit 72 is disposed dimensioned and configured to retain, direct and appropriately position the viewing device 30 and the wire harness 31 associated therewith. The conduit 72 may terminate approximately at the junction of the end segment 70, with the distal end 17 or may continue through the channel 14 of the tubular base 12. It is to be further noted that in one embodiment the wire harness 31 is a waterproof construction. As such the wire harness 31 can be retained in the manner represented by a hub or similar type connection at opposite ends thereof to maintain an appropriate alignment.

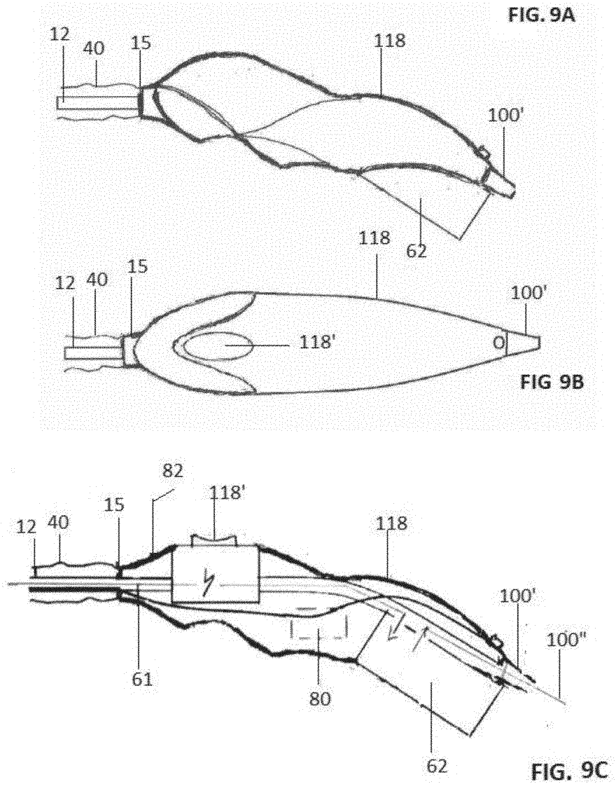

Moreover, in at least one embodiment to be described in greater detail hereinafter and schematically represented in FIG. 9C, the wire harness 31 can extend through the handle 118 and be connected to an onboard image processing unit 80 or a removable monitor or mobile communication device, such as a smart phone, via an optional mount or connection 82. In at least one additional embodiment the wire harness 31 can be effectively eliminated by structuring and implementing a wireless connection, wherein the viewing structure 30 can be modified to transmit a wireless signal to the onboard image processing 80.

As represented in FIGS. 3A-3C the inlet port 24 may be located in a different position from beveled and/or angled orientation and position as represented in FIG. 3. More specifically FIG. 3A discloses the inlet port 24 having a configuration which is somewhat aligned or parallel to the line of sight 300 of the viewing structure 30 but laterally spaced a sufficient distance, as at 400 so as not to interfere with the line of sight 300 of the viewing structure 30. FIG. 3B is representative of another structural modification wherein a plurality of inlet port 24 and 24″ are both disposed in communicating relation with the flow path 22 of the tip 20.

Also, at least one embodiment of the suction catheter assembly 10 of the present invention includes the flexible, deformable material, as described herein, extending along at least a portion of the length of the tip 20, and becoming progressively less flexible or deformable towards the distal end 17 of the base 12, to which the tip 20 is connected. In addition, in each of the structural modifications defining one or more embodiments of the present invention, the flexible, deformable material includes an inherent “memory” as set forth herein, which facilitates the tip 20 including the periphery of the periphery 24′ of the inlet port 24 returning to at an original shape, dimension and intended operative orientation. as at least partially represented in FIGS. 1A and 2 A. Moreover, at least some portion of the length of the tip 20, from and including the outwardly or distally disposed inlet port 24, towards the proximal end 15 of the base 12, may be formed of a material including the “memory” and flexible, temporarily deformable characteristics of the type set forth above. As should be noted, the memory capabilities allow a reorientation of the initial or original angular orientation of the tip 20 upon being exposed to an external force and would therefore allow the tip 20 to be effectively straightened to facilitate passage of the catheter through “straight” conduits. Thereafter, upon removal of such an external force the tip 20 would regain its preferred and original angled or deflected or offset orientation such as, but not limited to, that represented in FIG. 3C. Therefore, the original angular orientation or overall configuration of the tip 20 as represented in FIG. 3C is at least partially defined by an outside radius 20″ comprising a gradual curve segment 20′, rather than a sharp angled bend.

Moreover, when the tip 20 is deformed, such as by when exposed to an external force, the original angular orientation and/or deformed orientation when exposed to an external force, the tip 20 may assume an angular orientation of generally between about 5° and 90° relative to the length and/or axis of the tubular base 12, thereby incorporating any practical angle of orientation of the tip 20 which would be appropriate or required for clinical usage.

Also, it is to be further noted that the orientation of the inlet port 24, as at least partially defined by the surrounding periphery 24′ thereof, may be other than the angled or beveled orientation, as represented in FIG. 3-3C. Such additional orientations of the periphery 24′ may still be at least partially formed of the aforementioned flexible, temporarily deformable material. By way of example, additional operative embodiments of the suction catheter assembly 10 of the present invention may include the outer periphery 24′, which defines the inlet port 24 having a substantially flat orientation, as represented in at least FIGS. 1 and 2. As such, the surrounding outer periphery 24′ of the inlet port 24 is disposed in a plane which is substantially perpendicular to the central axis of the flow path 22 of the tip 20.

As at least generally set forth above, factors associated with a successful collection of secretions, etc. from within a body lumen or medical tubing at least partially depends on the accurate locating of the secretions, etc. to be collected. As is recognized in the prior art, the asymmetric configuration or disposition of the primary branches of the lungs tend to direct a catheter to and through the right branch thereof. Therefore, the ability to “steer” the suction catheter assembly 10, in each of its embodiments, will render its operation significantly more efficient. As also noted herein, steering factors associated with known or conventional medical instruments such as, but not limited to a bronchoscope, involve relatively complex and or bulky steering structure.

Therefore, one operative and structural feature of the suction catheter assembly 10 of the present invention includes the ability to position the inlet port 24 of the tip 20 in a desired location on the interior of the body lumen or medical tubing by integrated steering capabilities, which are distinguishable from add-on structural and operative components to accomplish steering. Such integrated steering capabilities eliminate the necessity of having numerous and sometimes complex steering components of the type normally associated with prior art catheters and/or bronchoscope structures. More specifically and with primary reference to FIGS. 5, 5A and 5B at least one embodiment, the base 12 as well as it's connection, such as at proximal end 15, of the suction catheter assembly 10 is formed of a material of at least partially rigid or of semi-rigid construction. More specifically, at least the tubular base 12 has sufficient rigidity to facilitate steering thereof by allowing a rotation of the length of the base 12, such as by a rotational force 500, being applied to the proximal end 15 and tubular base 12, through manipulation of the handle or control hub 18, connected thereto.

As indicated, the sufficient rigidity of the at least tubular base 12 also facilitates a longitudinally directed “pushing” force being applied thereto so as to position it along the length of the body or artificial lumen. Moreover, this combination or variance and/or different gradients of rigidity along at least the tubular base 12 and to certain extent the tip 20, can be achieved through the use of different plastics or other appropriate materials, and/or varying thicknesses of the same plastic or materials, resulting in a construct of a catheter assembly 10 and/or at least the tubular body 12 with optimal push ability and rotational control at the proximal end and along at least a majority of the base 12, while preserving flexibility and reversible deformity (memory) of the preferentially deflected tip 20 at the distal end 17 of the base 12.

Moreover, in at least one embodiment of the catheter assembly 10 is structured to define a gradient of rigidity for the handle 18, base 12 and possibly the tip 20. The material or materials used in at least the tubular base 12 must further be capable of allowing flexibility of the base 12 as it bends away from the original, possibly straight-line orientation relative to the handle 18 in its resting state, without kinking. This is advantageous for preservation of the hollow interior channel 14 during clinical use. Further, in at least one embodiment a higher rigidity proximal portion, as at the proximal end 15, transitions after between at least 5 mm and up to 2 cm or possibly longer to a substantial midpoint of the tubular base 12. Thereafter or at an outer portion of the base 12, a lesser degree of rigidity extends to the distal end 17. Such variance or gradients in rigidity extending along the length of at least the tubular base 12 should be sufficient to facilitate rotation of the tip 20 through rotational manipulation of the handle or control hub 18, while still allowing sufficient flexibility to facilitate a bending or flexure of the tubular base 12 as it conforms to the interior configuration of the lumens, through which it passes. Such rigidity also facilitates an operator to “push” the catheter assembly 10, substantially longitudinally, through and along the length of a body or artificial lumen. However, such rigidity should be such as to not interfere with an appropriate amount of flexure or bending of at least the tubular base 12 so as to conform to the overall configuration of the lumen through which it passes.

As also indicated herein the tip 20 may be of a softer material in order to prevent damage to interior tissue of a human lumen and may be somewhat narrower than that of the tubular base 12, with a rounded extremity adjacent the perimeter 24′ and/or inlet port 24 and or extremity 25. As represented in FIGS. 5A and 5B, the display on a monitor 200 resulting from rotational manipulation of the tip 20 may disclose a concurrent viewing of a first airway “A” and the second airway “B”. The advantage of effective rotation of the tip, through rotational manipulation of the catheter, specifically including the at least semirigid tubular base 12 is demonstrated. More specifically FIG. 5A disclose a concurrent viewing of the airways A and B such that the location of the viewing device 30 facilitates a concurrent viewing of the tip 20 thereby providing adequate orientation. To further add in the perceptive orientation of the tip, a marker or other indicator 205 may be located on the monitor itself. As a result, a 90° counterclockwise rotation 500 (see FIG. 5) of the tip in order to provide a better view of airway A is represented in FIG. 5B such that the tip 20 is more aligned with the airway A which in turn is aligned with the marker 205. If the tip 20 were deflected away from the line of sight or field-of-view on the monitor, the operator would retain awareness of the location of the tip 20 by way of marker or indicator 205.

Therefore, the application of such rotational force 500 of the base 12 will result in the axial rotation of the length thereof. In cooperation therewith, the laterally outward spacing 400 (see FIGS. 2-3B) of at least a portion of the length of the tip 20 and the inlet port 24, relative to the central axis of the base 12 the viewing structure 30, will result in an effective rotational pattern 600 of the tip 20. Such a rotational pattern 600 of the tip 20 and inlet port 24 provides for a rotational displacement of the tip 20 and inlet port 24, through 360° as represented in FIG. 5. In turn, this facilitates the tip 20 and inlet port 24 being variably and selectively positioned on the interior of the body lumen or medical tubing, upon the rotational force 500 being applied to the base 12. Therefore, such axial rotation of the length of the base 12 facilitates a steering or preferred rotational displacement of the tip 20 and the inlet port 24 so as to enter a desired branch, such as a primary or upper branch of the bronchial area. Once positioned therein, further axial rotation of the base 12 can be performed to affect a re-location location of the tip 20 and inlet port 24 to accomplish placement or positioning thereof to facilitate the collection of secretions, substances, etc. in the lower branches of the bronchial system. Further, accurate placement and/or steering is further facilitated aided by an illuminated viewing thereof by the illuminated viewing along the line of sight 300 generated by the viewing structure 30.

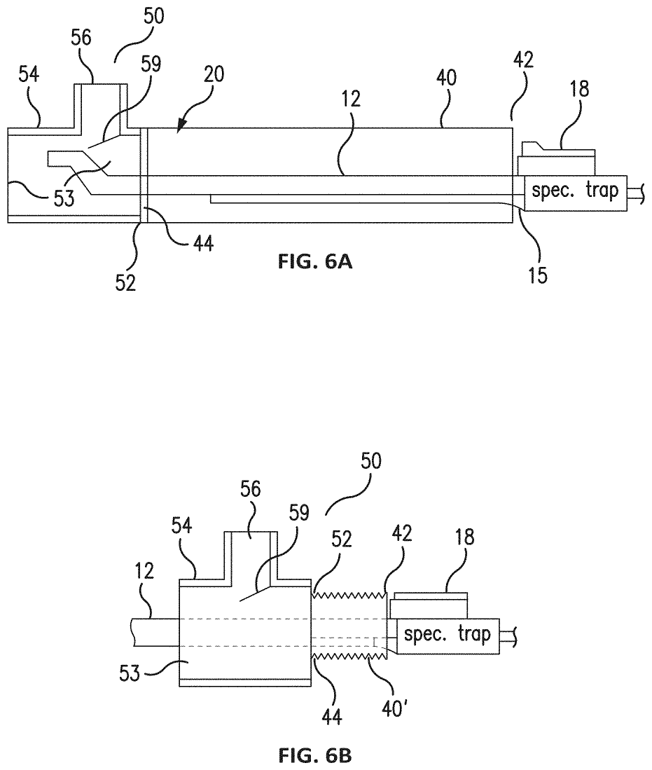

As represented in FIGS. 6A and 6B, yet additional features of the suction catheter assembly 10 of the present invention includes structure associated with maintaining sterility thereof prior to and after utilization thereof. It is of note that the catheter assembly 10 as represented in these figures may be structurally and operationally equivalent to anyone of the embodiments represented in FIGS. 1-8.

In more specific terms, and with reference to FIGS. 6A and 6B, one or more embodiments of the present invention may include an elongated, flexible and collapsible material sleeve 40 connected to the catheter assembly. The sleeve 40 has a sufficient length and/or elongated configuration extend along the length of and enclose a portion of the catheter assembly 10, including base 12 and tip 20, which extends into and through the intended body lumen or medical tubing, via an introductory coupler 50. As such, the sleeve 40 includes a proximal end 42 fixedly connected to a portion of the catheter assembly 10, such as at a proximal end 15 of the base 12. The opposite or distal end 44 of the sleeve 40 is preferably connected to the introductory coupler 50, which as explained in greater detail hereinafter may be considered an original component of the catheter assembly 10 or be used and initially provided independently thereof.

As is common practice, the catheter assembly 10 will be initially packaged and provided to medical personnel in a sterile condition. When removed from the packaging the elongated sleeve 40 will be disposed in its enclosing, covering position relative to a majority of the catheter and/or at least a portion thereof which is inserted into and through the body lumen or medical tubing via the introductory coupler 50. Accordingly, immediately prior to use and introduction of the catheter assembly 10 into the body lumen or medical tubing, the overlying, covering relation of the sleeve relative to the base 12 and the tip 20 will maintain the sterility thereof as originally established during or prior to packaging of the catheter assembly 10.

The opposite or distal end 44 of the sleeve 40 is preferably connected to the introductory coupler 50, by a flexible sealing member “S” such as, but not limited to, a washer-type structure (see FIG. 1C) to keep pressurized air from traversing from the coupler 52 the sterile sleeve 40. The distal end 44 is maintained in such attached or connected disposition during insertion and removal of the base 12 and tip 20 relative to the body lumen or medical tubing, via the introductory coupler 50. Accordingly, prior to use and insertion in the intended manner, the sleeve 40 and the catheter assembly 10 is in the operative position represented in FIG. 6A. As also noted, the introductory coupler 50 is cooperatively positioned at the insertion or introduction site and or is attached to additional medical instrumentation which facilitates accurate direction and insertion of the tip 20 and base 12 into the intended bronchial passage (not shown for purposes of clarity). As a result, the sterility of the catheter assembly 10, including the portions thereof, tip 20 and base 12, that are inserted through the introductory coupler and into the lumen of the body will be maintained in its initial sterile condition.

FIG. 6B is representative of the catheter assembly 10 while inserted and disposed in an operative position relative to the body lumen or medical tubing and the introductory catheter 50. While the catheter assembly 10, including base 12 and tip 20 are in such an inserted operative position, the flexible characteristics of the material from which the sleeve 12 is formed will allow it to be compacted, collapsed and/or folded upon itself, as at 40′, between its proximal and distal ends 42 and 44 respectively. As noted above, the proximal end 42 is fixedly connected to the catheter preferably, but not necessarily, adjacent the proximal end 15 of the base 12. In cooperation therewith, the distal end 44 of the sleeve 40 is fixedly attached or connected to the introductory coupler 50, which may be through sealing member “S” washer-like connector, as represented in FIG. 1C. Accordingly, concurrent to the insertion of the tip 20 and base 12 through the introductory coupler 50 and into the body lumen or medical tubing, the flexible sleeve 40 will begin to collapse or be folded upon itself as at 40′ in FIG. 6B.

However, subsequent to the intended drainage of aspirate or other substance from an intended bronchial passage, the base 12 and the tip 20 will be slowly withdrawn from the body lumen or medical tubing through and at least partially out of the introductory coupler 50. Upon such withdrawal of the catheter assembly 10, the sleeve 40 will then assume its original elongated orientation as represented in FIG. 6A. As a result, the base 12 and tip 20 and/or the portion of the catheter assembly 10 inserted into the body lumen or medical tubing and/or the introductory coupler 56 will be maintained in a sterile condition relative to an outside influence, other than exposure to the body lumen or medical tubing during insertion.

It is recognized that drainage of the bronchial area, or other area of the patient's body, may be repeatedly required over a given period of time. Therefore, the maintenance of sterility, except for exposure to the patient, of the base 12 and tip 20 allows reuse of the catheter assembly 10 and thereby results in a significant savings to a medical care facility and/or patient by eliminating the utilization of different drainage catheter assemblies for each of a possible plurality of repeated drainage procedures.

Yet additional structural features of the present invention include the aforementioned introductory coupler and or valve assembly 50, which includes a central passage 53, 54 disposed in interconnecting relation between the corresponding end 44 of the sleeve 40 as well as the tip and base 20 and 12 respectively of the catheter assembly 10. As is common, introductory couplers of the type disclosed and utilized may have a variety of different structural and operative configurations, as schematically represented in FIG. 1C. Accordingly, the introductory coupler and/or valve assembly 50 may also include a supplementary or auxiliary port or passage 56. As also noted herein, the tip 20 in the various embodiments of FIGS. 1 through 6A has a deflected, laterally offset disposition relative to the viewing structure 30. Such offset or non-axial relation of the tip 20 may result in it inadvertently coming into contact with interior structure of the coupler 50 and or entry into the supplementary pas sage 56.

Therefore, in order to eliminate or restrict such inadvertent passage of the tip 20 through the introductory coupler 50 and maintain its intended travel through the primary passage 53, 54, a guide member 59 will be disposed, dimensioned and configured to guide or direct appropriate travel of the tip 20 into and through the primary or central passage 53, 54, as represented in at least FIG. 6A.

Yet another embodiment of the present invention comprises a specimen or secretions trap or trap assembly generally indicated as 60 in FIG. 8, which is operatively structured to be used in combination with at least one or more embodiments of the catheter assembly 10. As such the trap assembly 60 includes a collection trap access port 62 which may be connected to or otherwise integrated with or into the handle or control valve 18. Upon operation of the catheter assembly 10, a flow of secretions/specimen 61 will travel along the interior conduit 14 of the base 12 and be received and directed by the trap access port 62 into container or other secretions/specimen collection structure 64. Operative features of this embodiment of the suction catheter assembly 10 will be such as to continue the flow of negative pressure as at 100″ through the appropriate conduit based on the influence of the negative pressure source 100. Upon completion of the procedure, a cap or like structure 66 seals the access port 62 so that additional secretions may be removed by the negative pressure source. A separate cap or like structure (not shown) may be used to cover the access port coupler 68 on the trap 64 in order to secure, retain and possibly transport the trap secretions/specimens as that 61′ on the interior the trap 64.

Yet another embodiment of the present invention is represented in FIG. 9A-9C and represents the catheter assembly 10 including a different structural configuration of the handle or control hub 118. However, it is of note that the handle 118 is operative in the manner represented in the embodiment of FIG. 1A-1C as set forth above. More specifically, handle or control hub 118 is attached to the distal end, 15 of the tubular base 12 and includes the aforementioned flexible, collapsible sleeve 40 extending from the proximal end 15 in covering relation to the base 12. The handle or control hub 18 also includes the control segment 118′ and the fluid port 100′ from which secretions may pass under the influence of negative pressure, as set forth above.

Also as represented in FIG. 9C and as at least generally set forth above, in the at least one embodiment schematically represented in FIG. 9C, the wire harness 31 (see FIGS. 7A and 7B) can extend through the handle 118 and be connected to an onboard image processing unit 80 or a removable monitor or mobile communication device, such as a smart phone, via an optional mount or connection 82. In at least one additional embodiment the wire harness 31 can be effectively eliminated by structuring and implementing a wireless connection, wherein the viewing structure 30 can be modified to transmit a wireless signal to the onboard image processing 80.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A suction catheter assembly structured to be steerable within a body lumen or medical tubing comprising:
an elongated base including a channel extending interiorly along a length thereof,
said base structured to connect said channel to a source of negative pressure,
said base including sufficient rigidity along a length thereof to facilitate at least a rotational steering thereof along and through a lumen,
a tip connected to a distal end of said base in laterally deflected, offset relation to a central axis of said base,
said tip formed of a flexible material having memory capabilities and including a flow path disposed in fluid communication with said channel, and
said tip including an inlet port disposed in fluid communication with said channel, via said flow path.

2. The suction catheter assembly as recited in claim 1 wherein said memory capabilities comprise an angular reorientation of said base, out of said laterally deflected, offset relation to a central axis of said base, concurrent to said tip being subjected to an external force.

3. The suction catheter assembly as recited in claim 2 wherein said memory capabilities further comprise said tip being realigned into an original laterally deflected, offset relation to the central axis of said base, concurrent to a removal of the external force.

4. The suction catheter assembly as recited in claim 1 wherein the said base includes a variable degree of rigidity along a length thereof from a proximal end to a distal end.

5. The suction catheter assembly as recited in claim 1 further comprising a handle connected to a proximal end of said base; said tip fixedly positioned relative to said handle concurrent to said laterally deflected, offset relation to the central axis of said base.

6. The suction catheter assembly as recited in claim 1 further comprising a trap assembly including an access port and a collection structure, said access port disposed in fluid communication with said channel and said collection structure and structured to direct secretions passing along a flow path of said channel into said collection structure.

7. The suction catheter assembly as recited in claim 6 wherein said access port is integrated into a handle of the catheter assembly in fluid communication with an interior of said channel and the source of negative pressure connected to said base.

8. The suction catheter assembly as recited in claim 1 further comprising a viewing structure disposed in substantially aligned, parallel relation to a central axis of said base.

9. The suction catheter assembly as recited in claim 8 wherein said viewing structure is mounted on said tip, along an inside radius thereof, adjacent a distal end of said base, said viewing structure oriented to define a line of sight disposed in laterally outward, spaced relation to a flow path within said channel and in substantially aligned, parallel relation to a central axis of said channel.

10. The suction catheter assembly as recited in claim 8 further comprising an end cap connected to a distal end of said base and including a conduit disposed within said channel and structured to retain a wire harness of said viewing structure therein.

11. The suction catheter assembly as recited in claim 1 further comprising a flexible material sleeve disposable between and extended covering relation to at lease said base and said tip and a collapsed, compact, non-covering relation to said base and said tip; said sleeve connected adjacent a proximal end of said base.

12. A suction catheter assembly structured to be steerable within a body lumen or medical tubing comprising:
an elongated base including a channel extending interiorly along the length thereof,
said base structured to connect said channel to a source of negative pressure,
said base including sufficient rigidity along a length thereof to facilitate at least a rotational steering thereof along and through a lumen,
a tip connected to a distal end of said base in laterally deflected, offset relation to a central axis of said base; said tip including a flow path disposed in fluid communication with said channel,
a viewing structure mounted on said base adjacent a distal end thereof and disposed in substantially aligned, parallel relation to a central axis of said base,
said viewing structure oriented to define a line of sight disposed in laterally outward, spaced relation to a flow path of said channel and in substantially aligned, parallel relation to a central axis of said channel, and
said tip including an inlet port disposed in fluid communication with said channel, via said flow path.

13. The suction catheter assembly as recited in claim 12 wherein said tip is formed of a flexible material having memory capabilities; said memory capabilities comprising an angular reorientation of said base, out of said laterally deflected, offset relation to said central axis of said base, concurrent to said tip being subjected to an external force.

14. The suction catheter assembly as recited in claim 13 wherein said memory capabilities further comprise said tip being realigned into an original laterally deflected, offset relation to the central axis of said base, concurrent to a removal of the external force.

15. The suction catheter assembly as recited in claim 12 further comprising an end cap connected to a distal end of said base and including a conduit disposed within said channel and structured to retain a wire harness of said viewing structure therein, on an interior of said channel.

16. The suction catheter assembly as recited in claim 12 wherein the said base includes a variable degree of rigidity along a length thereof from a proximal end to a distal end.

17. The suction catheter assembly as recited in claim 15 wherein said variable degree of rigidity being sufficient to facilitate said rotational steering and longitudinal steering of said base and said tip within and along a length of a body lumen.

18. The suction catheter assembly as recited in claim 12 further comprising a handle connected to a proximal end of said base; said tip fixedly positioned relative to said handle concurrent to said laterally deflected, offset relation to the central axis of said base.

19. The suction catheter assembly as recited in claim 12 further comprising a trap assembly including an access port and a collection structure, said access port disposed in fluid communication with said channel and said collection structure, said access port structured to direct secretions passing along said flow path and said channel into said collection structure.

20. The suction catheter assembly as recited in claim 18 further comprising a handle, said access port is integrated into said handle in fluid communication with an interior of said channel and the source of negative pressure connected to said base.

21. A suction catheter assembly structured to be steerable within a body lumen or medical tubing comprising:
an elongated base including a channel extending interiorly along the length thereof,
said base structured to connect said channel to a source of negative pressure,
said base including sufficient rigidity along a length thereof to facilitate at least a rotational steering thereof along and through a lumen,
a tip connected to a distal end of said base and including a flow path disposed in fluid communication with said channel,
a viewing structure mounted on said base adjacent a distal end thereof and disposed in substantially aligned, parallel relation to a central axis of said base,
said viewing structure oriented to define a line of sight in substantially aligned relation to a central axis of said channel,
a sleeve formed of a flexible material and disposable between an extended covering relation to at lease said base and said tip and a collapsed, compact, non-covering relation to said base and said tip;
said sleeve structured to facilitate a sterile environment of said base and said tip when in said extended covering relation, and
said tip including an inlet port disposed in fluid communication with said channel, via said flow path.
